(19) United States
(12) Patent Application Publication
(10) Pub. No.: US 2009/0005661 A1
(43) Pub. Date: Jan. 1, 2009

(54) Non-invasive blood component measuring device and non-invasive blood component measuring method

(75) Inventors: Toshiyuki Ozawa, Miki-shi (JP); Shigehiro Numada, Kobe-shi (JP)

Correspondence Address: BRINKS HOFER GILSON & LIONE, P.O. BOX 10395, CHICAGO, IL, 60610, US

(73) Assignee: Sysmex Corporation

(21) Appl. No.: 12/215,520

(22) Filed: Jun. 27, 2008

(30) Foreign Application Priority Data
Jun. 29, 2007 (JP) 2007-171875

(51) Int. Cl. A61B 5/1455

(52) U.S. Cl. 600/322

(57) ABSTRACT

A non-invasive blood component measuring device comprising: a light source section for irradiating a light to a blood vessel through a skin; an imaging section for imaging the irradiated blood vessel through the skin; and a controller, including a memory under control of a processor, the memory storing instructions enabling the processor to carry out operations, comprising: creating a concentration profile based on an image obtained by imaging the blood vessel with the imaging section; calculating a blood component concentration based on the concentration profile; acquiring a shape feature of the concentration profile; and correcting the blood component concentration based on the shape feature of the concentration profile is disclosed. A non-invasive blood component measuring method is also disclosed.

### RELATED APPLICATIONS

This application claims priority under 35 U.S.C. §119 to Japanese Patent Application No. JP2007-171875 filed Jun. 29, 2007, the entire content of which is hereby incorporated by reference.

### FIELD OF THE INVENTION

The present invention relates to a non-invasive blood component measuring device and a non-invasive blood component measuring method for percutaneously measuring a blood component to be measured without drawing blood from a living body.

### BACKGROUND

A method and a device for non-invasively measuring hemoglobin concentration without drawing blood from a subject have been conventionally proposed. U.S. Pat. No. 6,061,583 Publication discloses a device for illuminating a living body tissue including blood vessels with a light source and imaging a transmitted light image, extracting an image concentration distribution distributed across the blood vessel from the imaged image as a concentration profile of the image, cutting out a portion corresponding to the blood vessel from the extracted concentration profile at a baseline, and measuring the blood component based on the cutout profile as a “non-invasive blood examination device”.

The hemoglobin concentration is calculated using a peak height of the concentration profile as a ratio between a portion where blood exists and a portion where blood does not exist, and a distribution width (half-value width) of the concentration profile at the height of 50% of the peak as the width of the blood vessel. That is, if the cross section of a blood vessel is a perfect circle, the blood vessel diameter in the imaging direction and the blood vessel diameter in a direction orthogonal to the imaging direction become equal. Therefore, the hemoglobin concentration can be calculated by substituting the half-value width reflecting the blood vessel diameter in the direction orthogonal to the imaging direction with a distance the illumination light has moved through the blood, and performing a calculation process assuming the Law of Beer is approximately satisfied.

However, the cross section of the blood vessel is not necessarily always a perfect circle, and sometimes deforms due to various reasons. For instance, if blood is not sufficiently flowing through the blood vessel, the pressure of the blood flow weakens and the blood vessel constricts, whereby the cross section of the blood vessel becomes an ellipse rather than a perfect circle. If the external temperature is low or depending on the bend of the wrist in time of measurement, or if the peripheral blood vessel has disability, the blood flow volume tends to become insufficient, whereby the blood vessel constricts and the cross section of the blood vessel deforms.

In the invention disclosed in U.S. Pat. No. 6,061,583, the hemoglobin concentration is measured on the assumption that the cross section of the blood vessel is a perfect circle, and thus a correct measurement cannot be made if the cross section of the blood vessel deforms and a measurement error creates with the actual measurement value.

### SUMMARY OF THE INVENTION

The scope of the present invention is defined solely by the appended claims, and is not affected to any degree by the statements within this summary.

A first aspect of the present invention is, a non-invasive blood component measuring device comprising: a light source section for irradiating a light to a blood vessel through a skin; an imaging section for imaging the irradiated blood vessel through the skin; and a controller, including a memory under control of a processor, the memory storing instructions enabling the processor to carry out operations, comprising: creating a concentration profile based on an image obtained by imaging the blood vessel with the imaging section; calculating a blood component concentration based on the concentration profile; acquiring a shape feature of the concentration profile; and correcting the blood component concentration based on the shape feature of the concentration profile.

A second aspect of the present invention is, a non-invasive blood component measuring device comprising: a light source section for irradiating a light to a blood vessel through a skin; an imaging section for imaging the irradiated blood vessel through the skin; and a controller, including a memory under control of a processor, the memory storing instructions enabling the processor to carry out operations, comprising: creating a concentration profile based on an image obtained by imaging the blood vessel with the imaging section; and calculating a blood component concentration based on a peak height of the concentration profile, and a shape feature of the concentration profile.

A third aspect of the present invention is, a non-invasive blood component measuring method comprising the steps of: irradiating a light to a blood vessel through a skin and imaging the irradiated blood vessel through the skin; creating a concentration profile distributed across the blood vessel based on an image obtained by imaging the blood vessel; calculating a blood component concentration based on the concentration profile; acquiring a shape feature of the concentration profile; and correcting the blood component concentration based on the shape feature of the concentration profile.

A fourth aspect of the present invention is, a non-invasive blood component measuring method comprising the steps of: irradiating a light to a blood vessel through a skin and imaging the irradiated blood vessel through the skin; creating a concentration profile distributed across the blood vessel based on an image obtained by imaging the blood vessel; and calculating a blood component concentration based on a peak height of the concentration profile and a shape feature of the concentration profile.

### BRIEF DESCRIPTION OF THE DRAWINGS

FIG. 1 shows a schematic structure of a non-invasive blood component measuring device according to an embodiment;

FIG. 2 is a cross sectional explanatory view showing the non-invasive blood component measuring device shown in FIG. 1;

FIG. 3 is a top view showing the structure of the light source;

FIG. 4 shows a positional relationship of light emitting diodes arranged on a holding plate;

FIG. 5 is a block diagram showing a structure of a measurement unit;

FIG. 6 shows an example of a screen displayed when the non-invasive blood component measuring device is in a standby state;

FIG. 7 shows an example of a screen displayed when the non-invasive blood component measuring device is aligned with a blood vessel position;

FIG. 8 shows an example of a screen displayed when the non-invasive blood component measuring device completes a measurement;

FIG. 9 is a flowchart showing a measurement operation by the non-invasive blood component measuring device;

FIG. 10 is a view in which a rectangular region including an imaging region CR is coordinate divided into two-dimensional coordinates of x, y in a range of 0≦x≦640, 0≦y≦480;

FIG. 11 shows an example of a luminance profile (luminance profile PF) of pixels in the x direction at the predetermined y coordinate;

FIG. 12 illustrates a method for determining the position of a blood vessel;

FIG. 13 is a flowchart showing details of a measuring process of a hemoglobin concentration executed in step S11 of the flowchart shown in FIG. 9;

FIG. 14 shows a distribution of concentration D with respect to position X;

FIG. 15 shows a distribution of luminance B with respect to position X;

FIG. 16 shows a distribution of concentration D with respect to position X;

FIG. 17 shows explanatory view showing the calculation process of a distribution width at a cutout height H;

FIG. 18 shows a graph plotting the relationship between the kurtosis of the concentration profile and the distribution width when the flatness degree of the cross section of the blood vessel is changed step-wise;

FIG. 19 shows a graph plotting the actually measured value obtained from the blood cell counting device and the calculated value by the non-invasive blood component measuring device according to the present embodiment for the hemoglobin concentration of a plurality of subjects;

FIG. 20 shows the result of measuring the error between the hemoglobin concentration calculated by the non-invasive blood component measuring device according to the present embodiment while changing the bend of the wrist and the actually measured value obtained from the blood cell counting device for the hemoglobin concentration of a plurality of subjects; and

FIG. 21 is a flowchart showing details of a measuring process of a hemoglobin concentration according to another embodiment.

### DETAILED DESCRIPTION OF THE PREFERRED EMBODIMENTS

The preferred embodiments of the present invention are described hereinafter with reference to the drawings.

An embodiment of a non-invasive blood component measuring device of the present invention will now be described in detail with reference to the accompanying drawings.

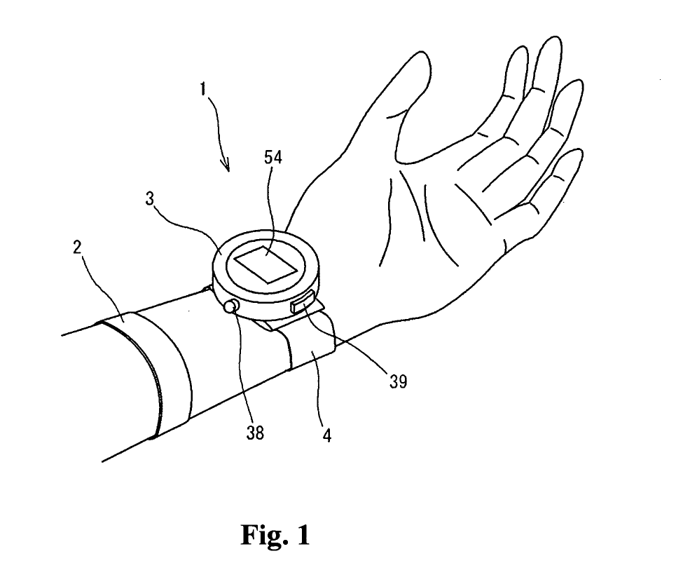

FIG. 1 shows a schematic configuration of a non-invasive blood component measuring device 1 according to a first embodiment of the present invention. The non-invasive blood component measuring device 1 is a wrist watch type device and includes a device body 3 and a holder 4. The device body 3 is attached to the wrist of a human by means of the holder 4. The device body 3 is attached in a position adjustable manner in a peripheral direction of the wrist by means of the holder 4. A power/execute key 38 and a menu key 39 for enabling the user to operate the non-invasive blood component measuring device 1 are arranged on the side face of the device body 3. A pressurization band 2 (cuff) is attached to the arm of the user closer to the heart than the wrist. The pressurization band 2 pressurizes the arm of the user at a predetermined pressure to inhibit the blood flow near the wrist, thereby dilating the blood vessel (vein) of the wrist. Thus, the imaging of the blood vessel is facilitated by making the measurement with the wrist being pressurized with the pressurization band 2.

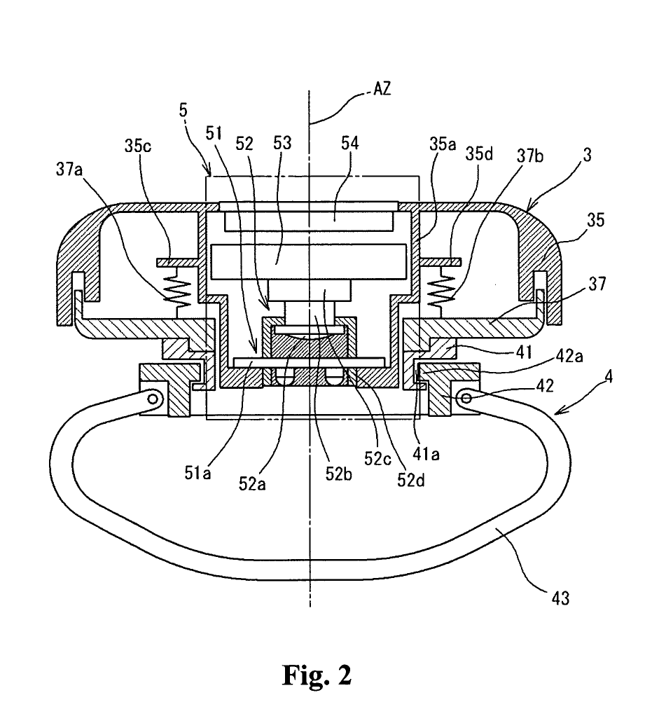

FIG. 2 shows a cross sectional explanatory view showing a configuration of the non-invasive blood component measuring device 1. The device body 3 includes an outer case 35, a back lid 37 arranged on the back side of the outer case 35, and an engagement member 41 attached to the lower part of the back lid 37. A cylindrical unit holding part 35a for accommodating a measurement unit 5, to be hereinafter described, is formed at the center of the outer case 35. A space part for receiving the unit holding part 35a is formed at the center of the back lid 37 and the engagement member 41. A pair of projections 35c, 35d are extending horizontally from an intermediate part of an outer wall of the unit holding part 35a. Compression springs 37a, 37b are connected between the projection 35c and the back lid 37, and between the projection 35d and the back lid 37, respectively. The outer case 35 is biased towards the back lid 37 by the compression springs 37a, 37b. An engagement part 41a depressed to a concave shape is formed at the side face of the engagement member 41 so as to be able to engage with an inner projection 42a of a supporting board 42 to be hereinafter described.

The holder 4 is configured by the supporting board 42 and a wrist band 43. The supporting board 42 has an upper surface shape of a rectangle, and has a circular opening to be fitted with the engagement member 41 of the device body 3 formed at a central part. The engagement part 42a to be rotatable engaged by the engagement member 41 about an axis AZ is formed at the edge of the opening. A stretchable rubber wrist band 43 is attached to the supporting board 42. The outer case 35 and the back lid 37 are made of material that does not transmit light.

The measurement unit 5 is supported by the unit holding part 35a. The measurement unit 5 is configured by a light source section 51, an imaging section 52, a controller 53, and a display section 54, wherein the light source section 51, the imaging section 52, the display section 54, and the controller 53 are connected by a wiring code, a flat cable (not shown), or the like so that electric signals can be mutually exchanged.

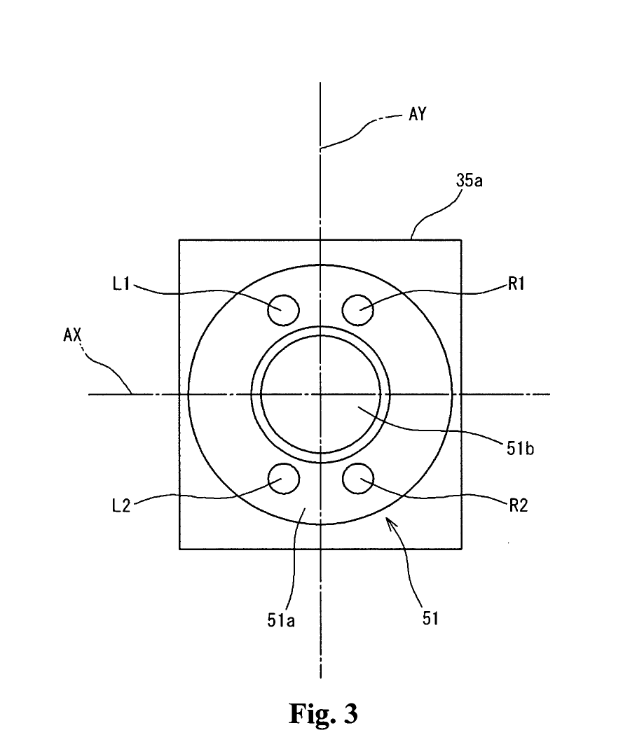

The light source section 51 will now be described. FIG. 3 is a top view showing the structure of the light source 51. The light source section 51 is configured by a circular plate shaped holding plate 51a, and four light emitting diodes R1, R2, L1, and L2 held by the holding plate 51a. A circular opening 51b for passing a light entering the imaging section 52 is formed at the center of the holding plate 51a, and the light emitting diodes are arranged along the periphery of the opening 51b.

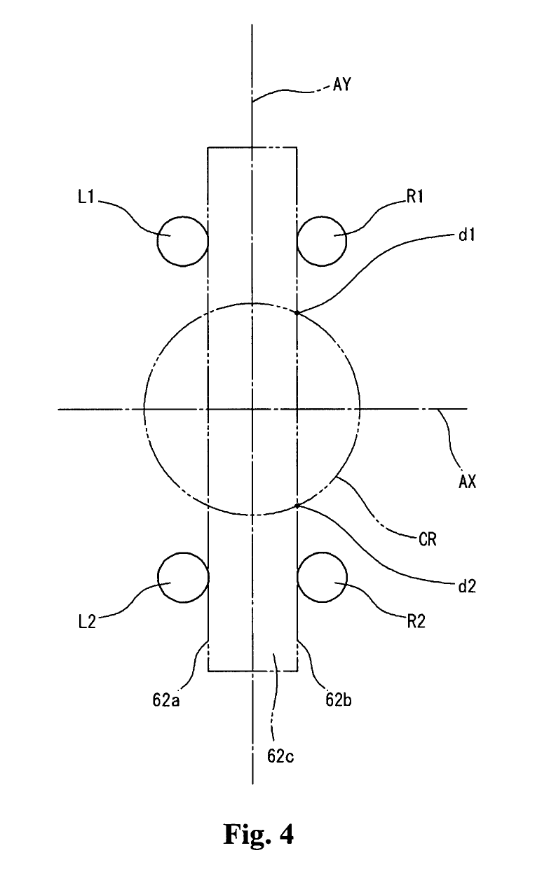

FIG. 4 shows a position relationship of the four light emitting diodes arranged on the holding plate 51a. The light emitting diodes R1, R2, L1, and L2 are arranged so as to be symmetric to a first axis AY and a second axis AX passing through the center of the opening 51b and being orthogonal to each other. In a state the non-invasive blood component measuring device 1 is attached to the wrist, an imaging region CR of the wrist surface is a region imaged by the imaging section 52, and displayed on the display section 54. A region 62c between an index line 62a on the light emitting diodes L1 and L2 (second light source section) side and an index line 62b on the light emitting diodes R1 and R2 (first light source section) side is the region suited for imaging by the imaging section 52, that is, the region where the blood vessel is to be positioned in time of imaging. The index lines 62a and 62b are displayed on the display section 54 by the controller 53. When analyzing the blood component, the attachment position of the device body 3 is adjusted so that an arbitrary blood vessel of the wrist is positioned within the region 62c. The blood vessel is illuminated with a near-infrared ray (center wavelength=805 nm) from both sides by the light emitting diodes R1, R2, L1, and L2.

The configuration of the imaging section 52 will now be described. As shown in FIG. 2, the imaging section 52 is configured by a lens 52a for narrowing the focus of a reflected light, a lens barrel 52b for fixing the lens 52a, and a CCD camera 52c for imaging, and is able to capture the image of the imaging region CR. The lens 52a and the lens barrel 52b are inserted to a cylindrical light shield tube 52d having a black interior portion. The Imaging section 52c capture the image, and transmits the same to the controller 53 as an image signal.

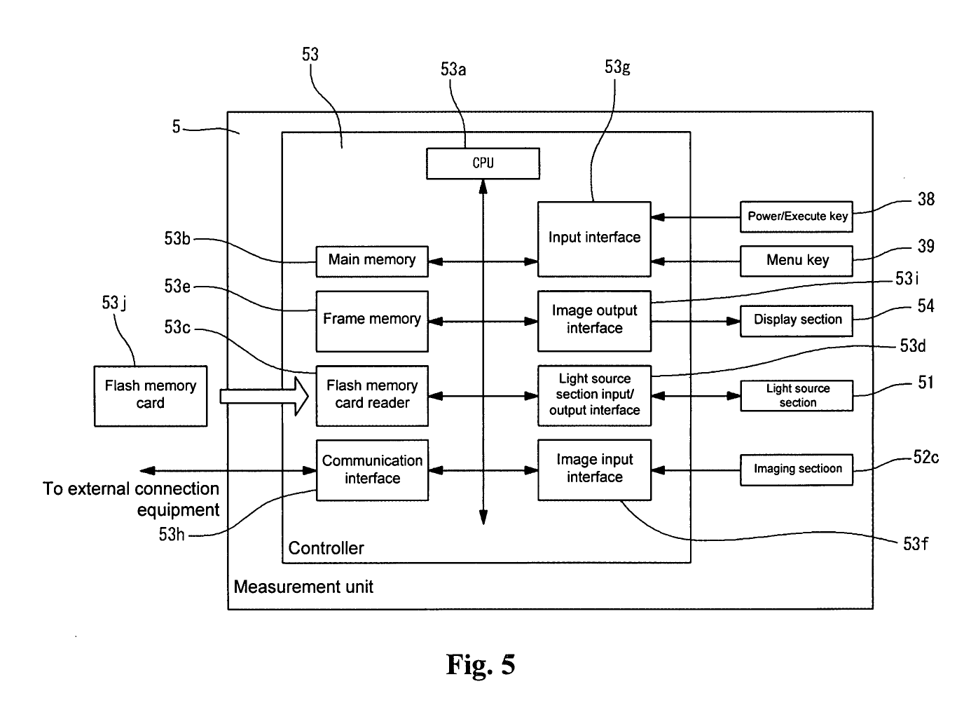

The configuration of the controller 53 will be described. The controller 53 is arranged on the upper part of the Imaging section 52c. FIG. 5 is a block diagram showing a configuration of the measurement unit 5. The controller 53 includes a CPU 53a, a main memory 53b, a flash memory card reader 53c, a light source section input/output interface 53d, a frame memory 53e, an image input interface 53f, an input interface 53g, a communication interface 53h, and an image output interface 53i. The CPU 53a, the main memory 53b, the flash memory card reader 53c, the light source section input/output interface 53d, the frame memory 53e, the image input interface 53f, the input interface 53g, the communication interface 53h, and the image output interface 53i are connected by way of a data transmission line so as to be able to mutually transmit data. According to such configuration, the CPU 53a can readout and write data with respect to the main memory 53b, the flash memory card reader 53c, and the frame memory 53e, and transmit/receive data with respect to the light source section input/output interface 53d, the image input interface 53f, the input interface 53g, the image output interface 53i, and the communication interface 53h.

The CPU 53a is capable of executing the computer program loaded in the main memory 53b. The present device functions as the non-invasive blood component measuring device when the computer program, as hereinafter described, is executed by the CPU 53a.

The main memory 53b is configured by SRAM, DRAM, or the like. The main memory 53b is used to read out the computer program stored in the flash memory card 53j. The main memory is also used as a work region of the CPU 53a when executing the computer programs.

The flash memory card reader 53c is used to read out the data stored in the flash memory card 53j. The flash memory card 53j includes a flash memory (not shown), and is able to hold the data without being supplied with power from the outside. The computer program executed by the CPU 53a, the data used for the same, and the like are stored in the flash memory card 53j.

An operating system complying with TRON specification is installed in the flash memory card 53j. The operating system is not limited thereto, and may be operating system providing graphical user interface environment such as Windows (registered trademark) manufactured and sold by US Microsoft Corp. In the following description, the computer program according to the present embodiment is assumed to operate on the operating system.

The light source section input/output interface 53d is configured by an analog interface including D/A converter, A/D converter, and the like. The light source section input/output interface 53d can be electrically connected with the four light emitting diodes R1, R2, L1, and L2 arranged in the light source section 51 by the respective electrical signal lines to perform the operation control of the relevant light emitting diode. The relevant light source section input/output interface 53d controls the current to be applied to the light emitting diodes R1, R2, L1, and L2 based on the computer program to be hereinafter described.

The frame memory 53e is configured by SRAM, DRAM, or the like. The frame memory 53e is used to store data when the image input interface 53f to be hereinafter described executes image processing.

The image input interface 53f includes a video digitize circuit (not shown) with an A/D converter. The image input interface 53f is electrically connected to the Imaging section 52c by an electrical signal line, so that image signals are input from the Imaging section 52c. The image signal input from the Imaging section 52c is A/D converted in the image input interface 53f. The image data digital converted as above is stored in the frame memory 53e.

The input interface 53g is configured by an analog interface including A/D converter. The power/execute key 38 and the menu key 39 are electrically connected to the input interface 53g. According to such configuration, the user can use the menu key 39 to select the operation item of the device, and use the power/execute key 38 to cause the device to turn ON/OFF the power of the device and to execute the operation selected by the menu key 39.

The communication interface 53h is configured by serial interface such as USB, IEEE1394, RS-232C; or parallel interface such as SCSI. The controller 53 can transmit and receive data with an external connection equipment such as mobile computer and portable telephone by using a predetermined communication protocol through the communication interface 53h. Thus, the controller 53 transmits measurement result data to the external connection equipment through the relevant communication interface 53h.

The image output interface 53i is electrically connected to the display section 54, and outputs the image signal based on the image data provided from the CPU 53a to the display section 54.

The display section 54 will now be described. As shown in FIG. 2, the display section 54 is arranged at the upper part of the measurement unit 5, and is supported by the outer case 35. The display section 54 is configured by a liquid crystal display, and performs a screen display according to the image signal input from the image output interface 53i. The screen display is switched according to the state of the non-invasive blood component measuring device 1, and for example, a screen corresponding to a measurement end state is displayed on the display section 54 in standby state, or in time of blood vessel alignment.

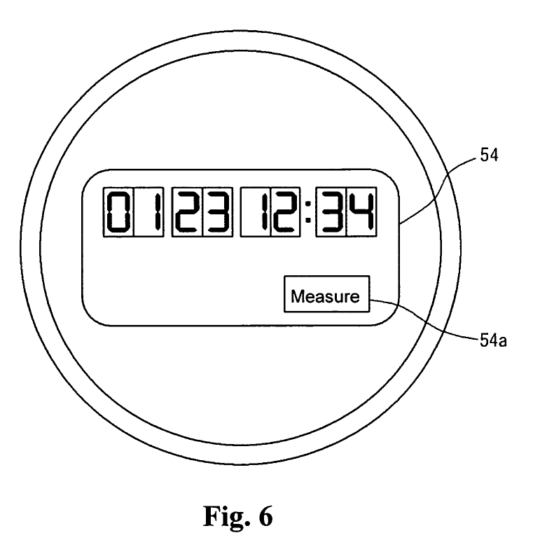

FIG. 6 shows one example of a screen displayed when the non-invasive blood component measuring device 1 is in the standby state. If the non-invasive blood component measuring device 1 is in the standby state, the date and the time are displayed at the center of the screen of the display section 54. A menu display region 54a is provided at the lower right of the screen of the display section 54, wherein the operation of the non-invasive blood component measuring device 1 of when the power/execute key 38 is pushed is displayed, and “measure” is displayed in the standby state.

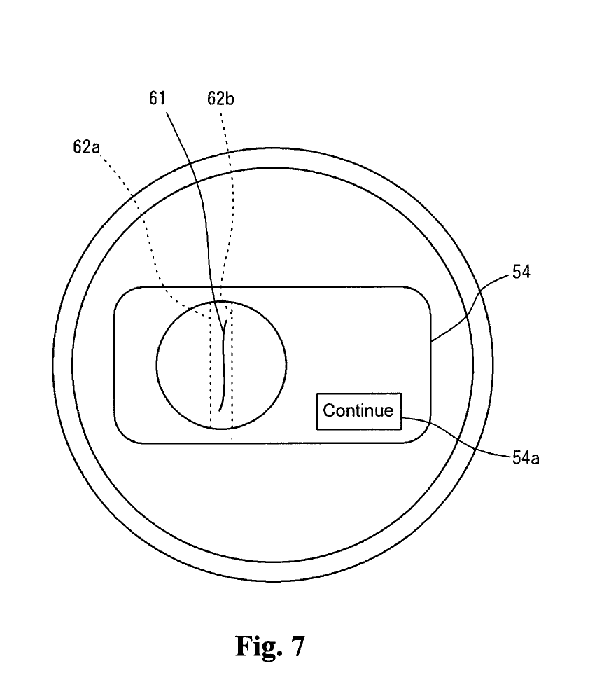

FIG. 7 shows an example of a screen displayed when the non-invasive blood component measuring device is aligned with a blood vessel position. The non-invasive blood component measuring device 1 according to the present embodiment is configured so that an index indicating a region suited for imaging by the imaging section 52 is displayed on the display section 54 and whether or not the blood vessel image is positioned within the region suited for imaging is determined. When aligning the blood vessel, a blood vessel pattern 61 formed as hereinafter described, and index lines 62a, 62b are displayed along with the image.

The index lines 62a and the index line 62b are displayed in red if the blood vessel pattern 61 is not positioned within the region 62c (see FIG. 4), and the index lines 62a and the index line 62b are displayed in blue if the blood vessel pattern 61 is positioned within the region 62c. The user then can easily understand whether or not the blood vessel pattern 61 is positioned within the region 62c.

According to such display, the user performs position adjustment by moving or rotating the device body 3 so that the blood vessel pattern 61 is within the region 62c.

In time of such blood vessel alignment, “continue” is displayed in the menu display region 54a, wherein when the blood pattern 61 is positioned within the region 62c, the index lines 62a, 62b are displayed in blue, the power/execute key 38 is validated, and the measurement is continued when the user pushes the power/key key 38.

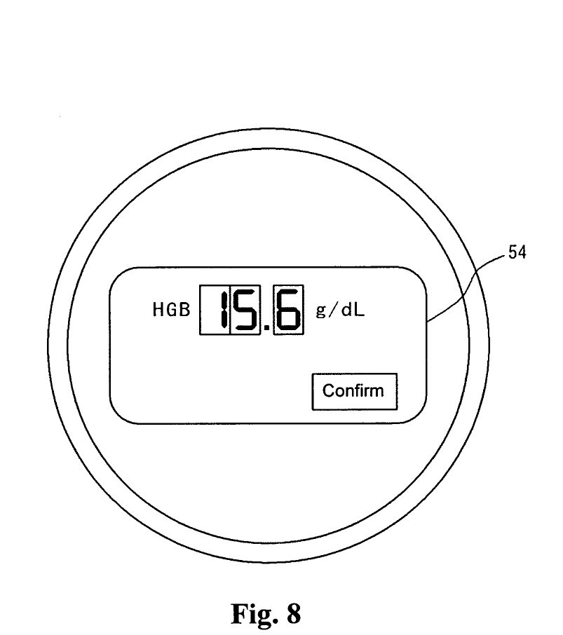

FIG. 8 shows an example of a screen displayed when the non-invasive blood component measuring device completes a measurement. The measurement result of hemoglobin concentration or blood component is displayed on the display section 54 in a digital representation as “15.6 g/dl” so as to be easily viewed by the user. “Confirm” is displayed on the menu display region 54a in this case.

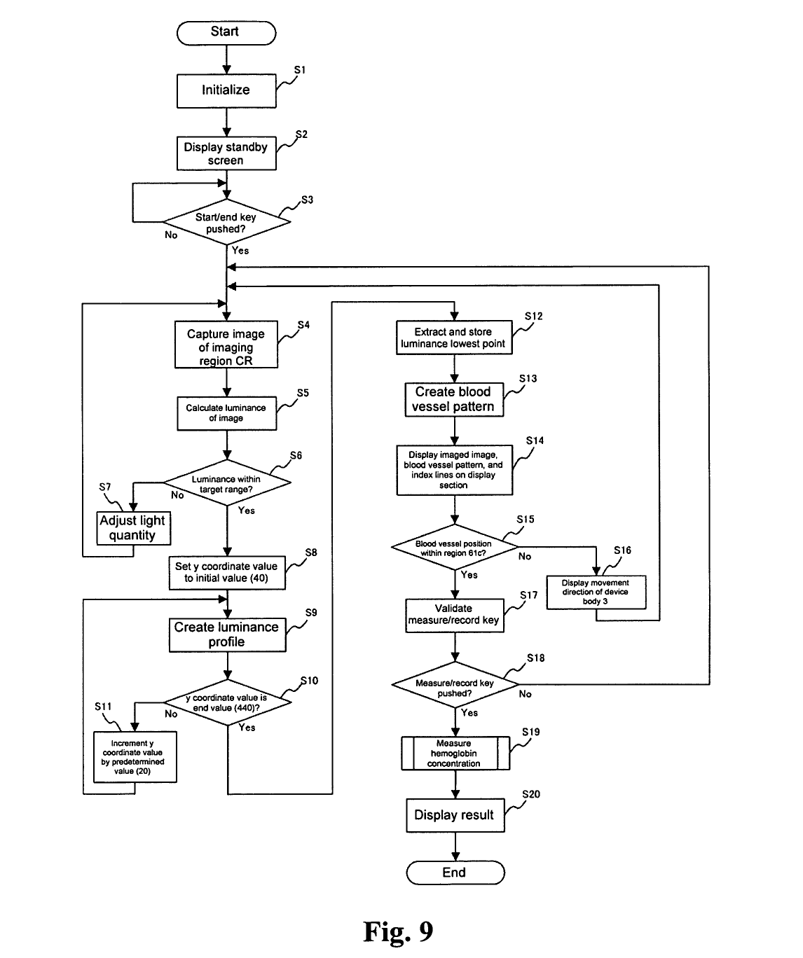

The measurement operation of the non-invasive blood component measuring device 1 will now be described. FIG. 9 is a flowchart showing the measurement operation by the non-invasive blood component measuring device 1. First, the pressurization band 2 is attached to the arm of the user, and the non-invasive blood component measuring device 1 is attached to the wrist (see FIG. 1). In this case, the arm of the user is pressurized with a predetermined pressure by the pressurization band 2, so that the blood flow near the wrist is inhibited and the blood vessels of the wrist are dilated. The user then pushes the power/execute key 38 arranged in the non-invasive blood component measuring device 1 to turn ON the power of the non-invasive blood component measuring device 1, so that initialization of the software is performed and the operation check of each unit is performed (step S1), whereby the device is in the standby state, and the standby screen (see FIG. 6) of the standby state is displayed on the display section 54 (step S2).

When the user pushes the power/execute key 38 while the screen of the standby state is being displayed on the display section 54 (Yes in step S3), the process proceeds to step S4.

The CPU 53a then lights the light emitting diodes R1, R2, L1, and L2 arranged in the light source section 61 respectively at a predetermined light quantity, illuminates the imaging region CR (see FIG. 4), and executes the process of capturing the image of the illuminated imaging region CR with an imaging section 52 (step S4). The captured image is stored in the frame memory 100e.

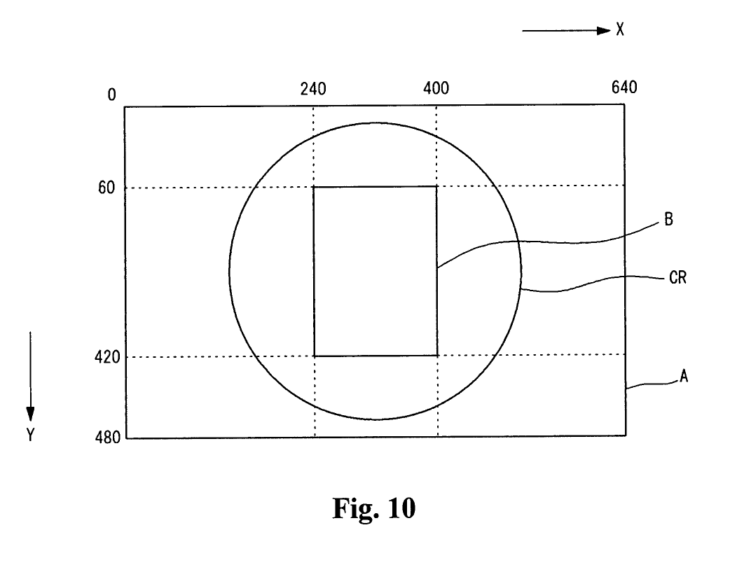

FIG. 10 is a view in which a rectangular region including the imaging region CR is coordinate divided into two-dimensional coordinates of x, y in a range of 0≦x≦640, 0≦y≦480. The CPU 53a coordinate divides the region A into two-dimensional coordinates of x, y with the coordinate of the most upper left pixel of the rectangular region A including the image of the imaging region CR as (0, 0), selects four points of (240, 60), (400, 60), (240, 420), (400, 420) from the coordinate divided points, and obtains an average luminance of a region B surrounded by the four points (step S5). The points of the region B for obtaining the average luminance are not limited thereto, and may be obviously other coordinates. The region B may be a polygon other than a square, or a circle.

The CPU 53a then determines whether or not the luminance of the region B is within a target range (step S6). If the luminance of the region B is outside the target range, the current amount flowing to the light emitting diodes R1, R2, L1, and L2 is adjusted using the light source section input/output interfaced 53d, the light quantity adjustment thereof is performed (Step S7), and the process returns to step S4. If the luminance of the region B is within the target range (Yes in step S6), the CPU 53a sets a y coordinate value to be calculated of the luminance profile to be hereinafter described to an initial value (40) (step S8). The luminance of the pixels from one end to another end of the x coordinate at the set y coordinate value (40) is obtained to create a luminance profile (step S9).

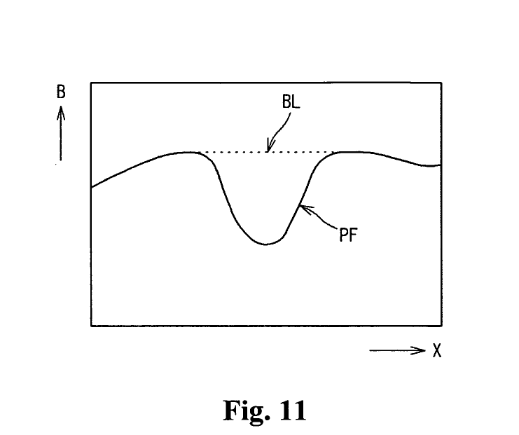

FIG. 11 shows one example of the luminance profile (luminance profile PF) of the pixel in the x direction at the predetermined y coordinate. When the luminance is obtained from the processes, the luminance profile (luminance profile PF) of the pixel in the x direction at the predetermined y coordinate is obtained. The CPU 53a then determines whether or not the set y coordinate value is an end value (440) (step S10). If the y coordinate value is not the end value (440) (No in step S10), the CPU 53a increments the y coordinate value by a predetermined value (20) (step S11), and returns the process to step S9. If the y coordinate value is the end value (440) (Yes in step S10), the CPU 53a extracts a point where the luminance is the lowest (hereinafter referred to as “luminance lowest point”) in each extracted luminance profile, and stores the same in the frame memory 53e (step S12).

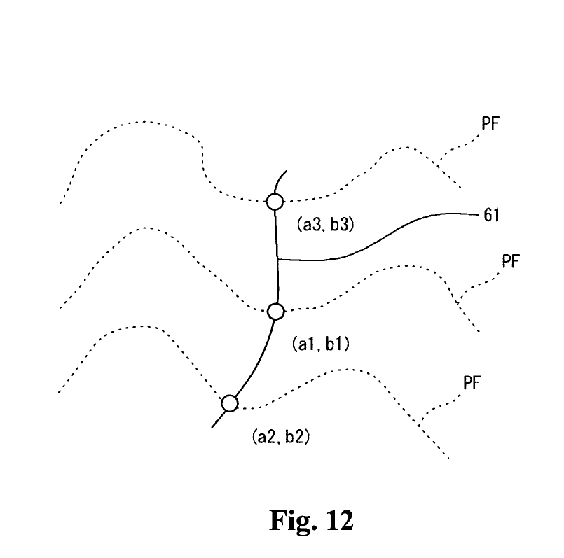

FIG. 12 illustrates a method for determining the position of a blood vessel. In order to obtain the position of the blood vessel, the CPU 53a connects the luminance lowest point (a1, b1) near the center of the image of the imaging region CR and the luminance lowest points (a2, b2) and (a3, b3) adjacent in the vertical direction of the luminance lowest point (a1, b1). The CPU 53a connects the luminance lowest point (a2, b2) and the point adjacent in the vertical direction, and connects the luminance lowest point (a3, b3) and the point adjacent in the vertical direction. The CPU 53a repeats this operation over the entire region of the image, extracts the blood vessel as a line segment column, and forms the blood vessel pattern 61 (step S13). The CPU 53a executes a process of displaying the image of the imaging region CR retrieved in step S4, the blood vessel pattern 61 formed in step S5, and the index line 62a and the index line 62b stored in the flash memory card 100j on the display section 54 (step S14). The CPU 53a determines whether or not the blood vessel pattern 61 is positioned in the region 62c (see FIG. 4) (step S15). If the blood vessel pattern 61 is not positioned within the region 62c (No in step S15), the COU 53a executes a process of instructing which direction the user should move the device body 3 (step S16). After the process of step S16 is terminated, the CPU 53a returns the process to step S4, and the CPU 53a again retrieves the captured image of the imaging region CR, and executes the processes of step S4 to S15. From the retrieval of the captured image of the imaging region CR in step S4 to the determination process of step S15 are performed on 1/100 seconds, and the display of the display section 54 is updated on 1/100 seconds scale. These processes are repeatedly executed while position adjustment is being carried out by the user, wherein the user adjusts the attachment position of the device while checking the display of the display section 54 that is updated as needed. The processes of steps S4 to S16 are repeated from when the position adjustment is carried out by the user until determined that the blood vessel pattern 61 is positioned within the region 62c by the CPU 53a.

When the CPU 53a determines that the blood vessel pattern 61 is positioned within the region 61c as a result of position adjustment by the user (Yes in step S15), the CPU 53a validates the power/execute key 38, and enables the measurement to continue (step S17). The CPU 53a then determines whether or not the power/execute key 38 is pushed by the user (step S18). If determined that the power/execute key 38 is not pushed, the CPU 53a returns the process to step S4, executes the processes of steps S4 to S14, and again determines whether or not the blood vessel pattern 61 is positioned within the region 61c in the process of step S15.

In the process of step S19, when the CPU 53a determines that the power/execute key 38 is pushed (Yes in step S18), the CPU 53a executes a process of hemoglobin concentration measurement (step S19). Once the measurement is terminated, the CPU 53a displays a measurement result display screen as shown in FIG. 8 on the display section 54 (step S20) and terminates the process.

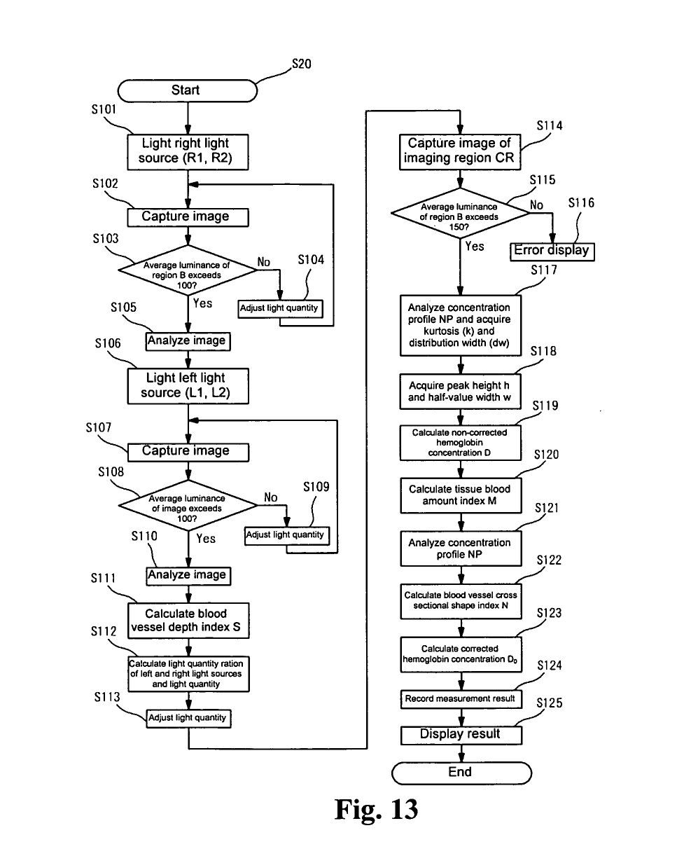

FIG. 13 is a flowchart showing details of the measuring process of hemoglobin concentration executed in step S19 of the flowchart shown in FIG. 9. When the power/execute key 38 is pushed, the CPU 53a controls the light source section input/output interface 53d, illuminates the living body containing the blood vessel at an appropriate light quantity by the light emitting diodes R1, R2 (first light source section), which is one of the light sources arranged on both sides with the blood vessel in between, (step S101), and captures an image of the same in the imaging section 52 (step S102). The CPU 53a determines whether or not the average luminance of the region B exceeds 100 (step S103), adjusts the current amount flowing to the light emitting diodes R1, R2 by using the light source section input/output interface 53d if the luminance does not exceed 100, and performs the light quantity adjustment thereof (step S104), and returns the process to step S102.

The value of luminance referred to herein is the digital conversion value (changes between 0 and 255) of the A/D converter of eight bits of the image input interface 53f being used in the present embodiment. This is because the luminance of the image and the magnitude of the image signal input from the Imaging section 52c are proportional, and thus the A/D conversion value (0 to 255) of the image signal is assumed as the value of luminance.

If the average luminance of the region B exceeds 100 (Yes in step S103), the CPU 53a obtains the luminance profile PF1 and the concentration profile NP1 non-dependent on the incident light quantity for the image obtained in step S102 (step S105). Furthermore, the CPU 53a controls the light source section input/output interface 53d, illuminates the living body containing the blood vessel at an appropriate light quantity by the light emitting diodes L1, L2 (second light source section), which is the other of the light sources arranged on both sides with the blood vessel in between, (step S106), and captures an image of the same in the imaging section 52 (step S107). The CPU 53a determines whether or not the average luminance of the region B exceeds 100 (step S108) and increases the current amount flowing to the light emitting diodes L1, L2 by using the light source section input/output interface 53d if the luminance does not exceed 100, performs the light quantity adjustment thereof (step S109), and returns the process to step S107.

If the average luminance of the region B exceeds 100 (Yes in step S108), the CPU 53a performs a process similar to step S105 for the image obtained in step S107, and obtains the luminance profile PF2 and the concentration profile NP2 non-dependent on the incident light quantity (step S10).

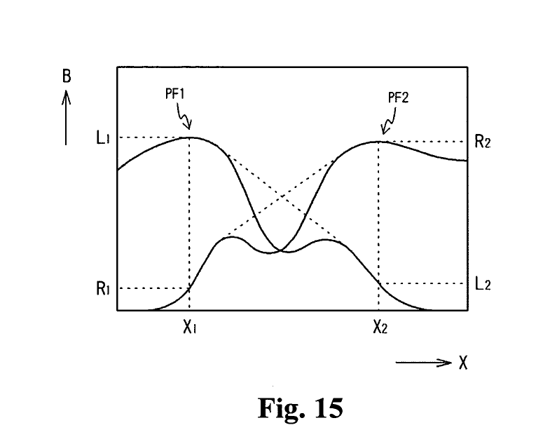

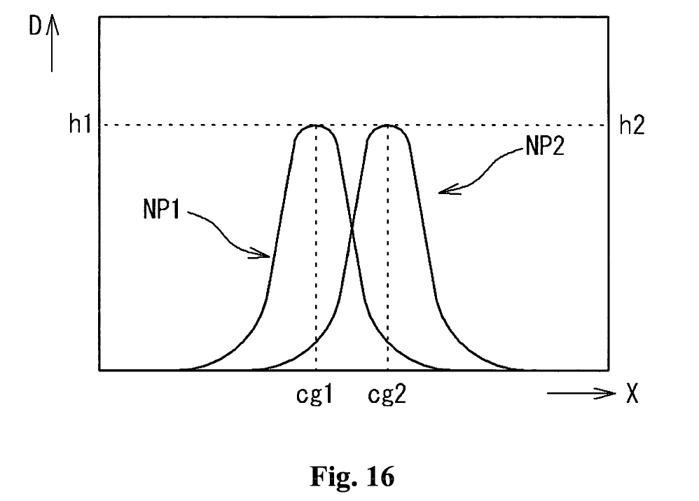

FIG. 15 shows a distribution of the luminance B with respect to the position X, wherein the luminance profile PF1 is formed by step S105 and the luminance profile PF2 is formed by step S110. FIG. 16 shows a distribution of the concentration D with respect to the position X, wherein the concentration profile NP1 is formed by step S105 and the concentration profile NP2 is formed by step S110.

The CPU 53a derives the peak value h1 and the barycentric coordinate cg1 from the concentration profile NP1 obtained by step S105, and the peak value h2 and the baryceritric coordinate gc2 from the concentration profile NP2 obtained by step S110, and calculates a blood vessel depth index S by using the above with the following calculation formula (1). Furthermore, the CPU 53a stores the calculation result in the frame memory 53e (step S111).

S=(cg2−cg1)/{(h1+h2)/2} (1)

The CPU 53a calculates the light quantity ratio of the left and right light sources (light emitting diodes R1, R2 and light emitting diodes L1, L2) of the blood vessel, and the light quantity based on the luminance profile PF1 obtained by step S105 and the luminance profile PF2 obtained by step S110 (step S112), and performs light quantity adjustment of both light sources based on the obtained result (step S113).

The CPU 53a then controls the light source section input/output interface 53d, illuminates the imaging region CR (see FIG. 4) with the light quantity adjusted light emitting diodes R1, R2, L1, and L2, and captures an image of the same in the imaging section 52 (step S114). The CPU 53a then obtains the average luminance of the region B shown in FIG. 10, and determines whether or not the obtained average luminance of the region B exceeds 150 (step S115). An error display is made if the luminance does not exceed 150 (step S116).

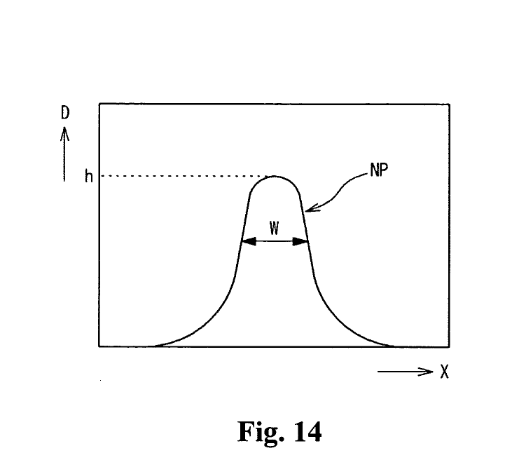

If the average luminance of the region B exceeds 150 (Yes in step S115), the CPU 53a creates a luminance profile (distribution of luminance B with respect to position X) PF (see FIG. 11) showing a first luminance distribution with respect to an axis AX in the imaging region CR (see FIG. 4), and reduces the noise by using methods such as fast Fourier transformation. The CPU 53a also standardizes the luminance profile PF with base line BL. The base line BL is obtained based on the shape of the luminance profile of the absorption portion by the blood vessel. The concentration profile (distribution of concentration D with respect to position X) NP non-dependent on the incidence light quantity is thereby created (step S117). FIG. 14 shows a distribution of the concentration D with respect to the position X, and the concentration profile NP as shown in the figure is created.

The CPU 53a calculates a half-value width was the distribution width corresponding to the peak height h and the blood vessel diameter based on the created concentration profile NP (step S118). The half-value width w is the distribution width at 50% of the peak height of the concentration profile NP. The peak height h represents the ratio of the light intensity absorbed by the blood vessel (blood) to be measured and the light intensity passed through the tissue portion, and the half-value width w represents the length corresponding to the blood vessel diameter in the direction orthogonal to the imaging direction. The CPU 53a then calculates a non-corrected hemoglobin concentration D with the following formula (2), and stores the result in the frame memory 53e (step S119).

D=h/wⁿ (2)

Here, n is a constant representing non-linearity of the spread of the half-value width due to scattering. If there is not light scattering, n=1, and if there is scattering, n>1.

The CPU 53a calculates a tissue blood amount index M representing the blood amount contained in the peripheral tissue based on the blood vessel peripheral tissue image in the image of the living body obtained in step S101 (step S120). Specifically, a second luminance distribution distributed along the blood vessel image is extracted based on the blood vessel peripheral tissue image in the image of the living body at a predetermined distance (e.g., 2.5 mm) from the blood vessel image in the image of the living body. The portion that seems to be saturated of the second luminance distribution is eliminated, and only the portion that can be substantially assumed as a parabola is remained. The tissue blood amount index M including the attenuation rate of the light is obtained based on the following formula with y0 as the luminance of the end portion of the remaining portion, y1 as the luminance at the point of lowest luminance, and was the distance from one end to the other end.

$${( \frac{y 0 - \sqrt{y 0 \cdot y 0 - y 1 \cdot y 1}}{y 1} )}^{\frac{2}{W}}$$

The CPU 53a stores the obtained tissue blood amount index M in the frame memory 53e.

The CPU 53a then analyzes the hill shaped concentration profile NP created in step S117 (step S121), calculates a blood vessel cross sectional shape index N (step S122), and stores the calculation result in the frame memory 53e.

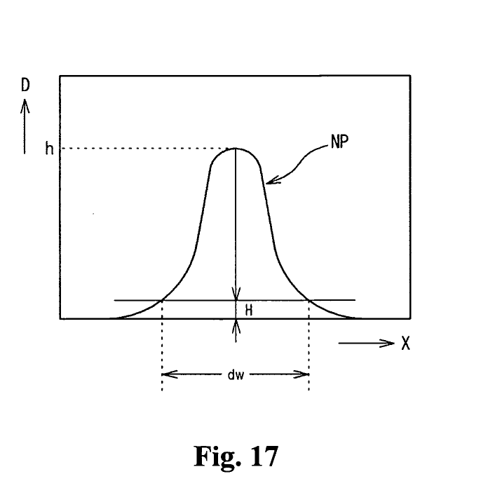

The is calculated in the following manner. First, a cutout height H is set with respect to the concentration profile NP obtained in step S117, the concentration profile NP in the cutout range is assumed as a distribution density function of a probability variable, and a kurtosis (k) in the function and a distribution width (dw) at the cutout height H are calculated. FIG. 17 shows explanatory view showing the calculation process of a distribution width (dw) at a cutout height H. The cutout height H is a percentage of the peak height h which determines the range of analyzing respect to the concentration profile NP for calculating the blood vessel cross sectional shape index N, as shown in the figure. The kurtosis (k) is obtained from the concentration profile NP existing above the cutout height H, and the distribution width (dw) is obtained from the distribution width (length of bottom) of the concentration profile NP in the cutout range. The cutout height H=0.01% is preferable.

The values of the kurtosis (k) and the distribution width (dw) obtained as above are substituted to the following formula (3) to obtain the blood vessel cross sectional shape index N.

N={(k+α)/dw^{β}}/(π·w²/4) (3)

Here, α and β are constants determined experimentally, and π is the circumference ratio. The blood vessel cross sectional shape index N and the formula (3) will be hereinafter described.

The CPU 53a obtains a correction coefficient fs based on the blood vessel depth index S calculated in step S111, a correction coefficient fm based on the tissue blood amount index M calculated in step S120, and a correction coefficient fn based on the blood vessel cross sectional shape index N calculated in step S122. The CPU 53a calculates the corrected hemoglobin concentration D₀ based on the following formula (4) by using such correction coefficients (step S123).

D₀=D×fs×fm×fn (4)

The CPU 53a stores the calculation result in step S123 in the frame memory 53e (step S124), executes the process of displaying the measurement result on the display section 54 as shown in FIG. 8 (step S125), and returns the process to the main routine.

In the present embodiment, the blood vessel depth index S, the tissue blood amount index M, and the blood vessel cross sectional shape index N are sequentially calculated, and the non-corrected hemoglobin concentration D is corrected at the point all the correction coefficients are calculated, but the configuration of the present invention is not limited thereto. For instance, a primary correction may be performed at the point the blood vessel depth index S is calculated, and the secondary correction may be performed at the point the tissue blood amount index M is calculated.

In the hemoglobin concentration measuring process according to the present embodiment, the kurtosis (k) and the distribution width (dw) are calculated after the non-corrected hemoglobin concentration D is calculated, but the order is not limited thereto. For instance, the non-corrected hemoglobin concentration D may be calculated after the kurtosis (k) and the distribution width (dw) are calculated.

The blood vessel cross sectional shape index N and the formula (3) will be described below. The blood vessel cross sectional shape index N is the index that indicates the shape of the blood vessel cross section. Here, the blood vessel cross sectional shape index N is defined as the ellipticity (ratio of diameter of minor axis with respect to diameter of major axis of an ellipse) of the blood vessel cross section under the assumption the blood vessel cross section is an ellipse. The blood vessel cross sectional shape index N is expressed by the following formula (5) where 2a is the blood vessel diameter in the imaging direction (direction of axis AZ in FIG. 2), and 2b is the blood vessel diameter in the direction orthogonal to the imaging direction (direction orthogonal to AZ axis in plan view in FIG. 2).

N=2a/2b=a/b (5)

The formula (3) for calculating the blood vessel cross sectional shape index N will now be described.

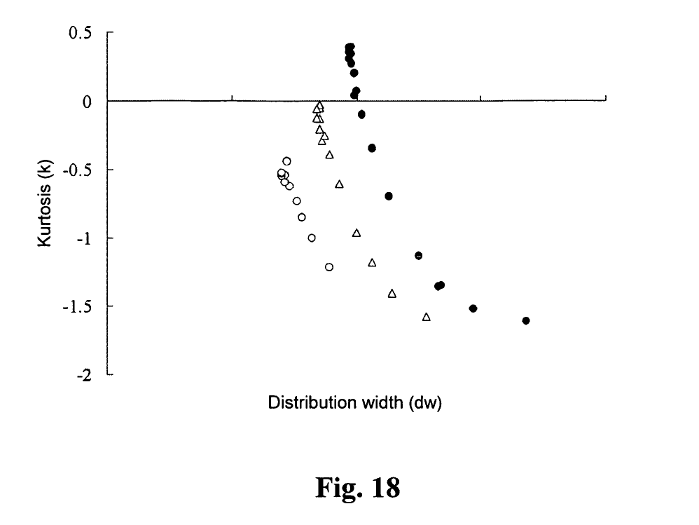

FIG. 18 is a graph plotting the relationship between the kurtosis (k) of the concentration profile NP and the distribution width (dw) at the cutout height H when the flatness degree of the blood vessel cross section is changed gradually with the cutout height H as 0.01% with respect to the concentration profile NP extracted based on three types of blood vessels having different cross sectional areas. The vertical axis is the kurtosis (k), the horizontal axis is the distribution width (dw), and the data related to the same cross sectional area is indicated with the same symbol.

As apparent from the figure, the kurtosis (k) and the distribution width (dw) change with drawing a constant correlation curve, unless the cross sectional area is changed, even if the flatness degree of the blood vessel cross section is changed. This means that, once the kurtosis (k) and the distribution width (dw) at the cutout height H are obtained, the cross sectional area Sa of the blood vessel to be measured can be estimated using the kurtosis (k) and the distribution width (dw) as indices.

Focusing on such aspects, in the present embodiment, the approximation formula based-on the correlation between the kurotsis k and the distribution width (dw) at the cutout height H is obtained as the following formula (6).

Sa=(k+α)/dw^{β} (6)

Here, α and β are constants determined experimentally.

From a different viewpoint, the area Sa of the blood vessel cross section of when the cross sectional shape of the blood vessel is an ellipse is obtained with the following formula (7).

Sa=π·a·b (7)

Thus, according to formula (6) and formula (7), the following formula (8) is obtained.

π·a·b=(k+α)/dw^{β} (8)

Solving the formula (8) so that the left side becomes a/b, the following formula (9) is obtained.

a/b={(k+α)/dw^{β}}/(π·b²) (9)

According to formula (5) and formula (9), the following formula (10) is obtained.

N=a/b={(k+α)/dw^{β}}(π·b²) (10)

Furthermore, in formula (10), the value b is the blood vessel radius in the direction orthogonal to the imaging direction, and the value b can be substituted by ½ of the half-value width w of the concentration profile NP. Therefore, following formula (11) is obtained.

N=a/b={(k+α)/dw²}/(π·w²/4) (11)

Then, that is proved that formula (3) is logical.

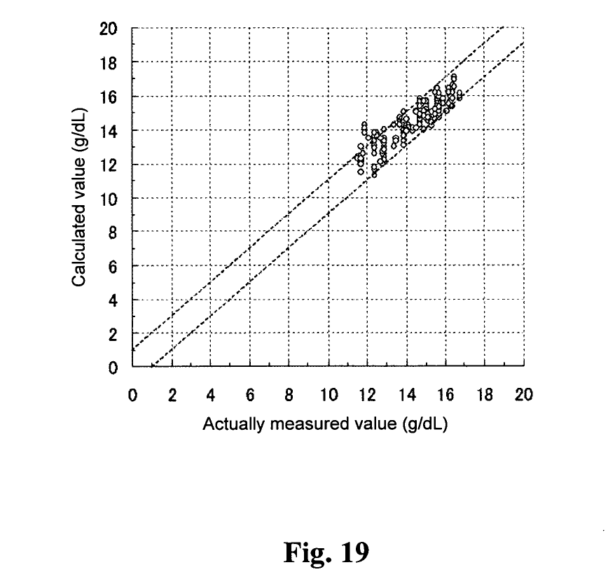

FIG. 19 is a graph plotting the actually measured value obtained from the blood cell counting device, and the calculated value by the non-invasive blood component measuring device 1 according to the embodiment of the present invention for the hemoglobin concentration of a plurality of subjects. As shown in the figure, the actually measured value and the calculated value by the non-invasive blood component measuring device 1 exist in the vicinity of a region surrounded by a line having a slope 1, and the actually measured value and the calculated value are not deviated. Then, it can be seen that the non-invasive blood component measuring device 1 can accurately measure the hemoglobin concentration.

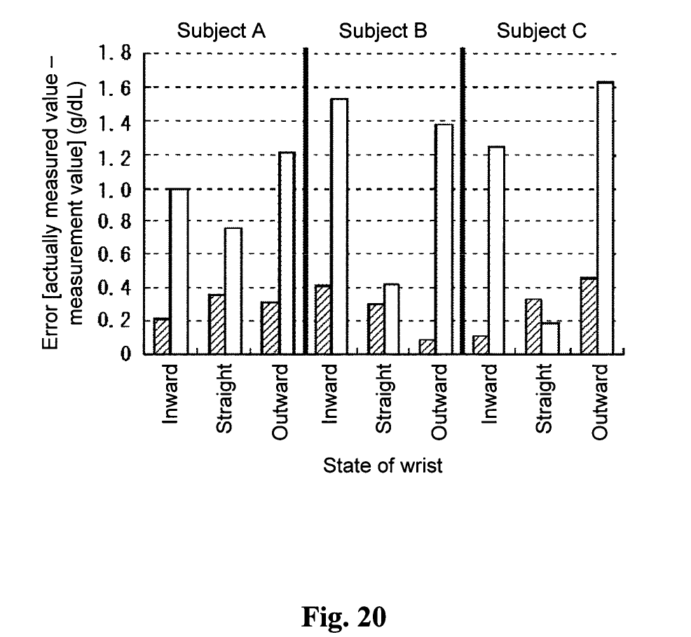

FIG. 20 shows the result of measuring the error between the hemoglobin concentration calculated by the non-invasive blood component measuring device 1 according to the present embodiment while changing the bend of the wrist in three ways (inward, horizontal, outward) and the hemoglobin concentration calculated by the conventional device. The shaded bar graph shows the result obtained by measuring with the non-invasive blood component measuring device of the present embodiment, and the outlined bar graph shows the result obtained by measuring with the conventional device. As apparent from the figure, the error with the actually measured value is suppressed within 1 g/dl even if the bend of the wrist is changed in various ways, and a measurement result without variation of measurement value is obtained even if the bend of the wrist is different. Therefore, it is verified that according to the non-invasive living body component measuring device of the present embodiment, an accurate and stable hemoglobin concentration measurement can be made even if the dilate state of the blood vessel is changed due to external factors.

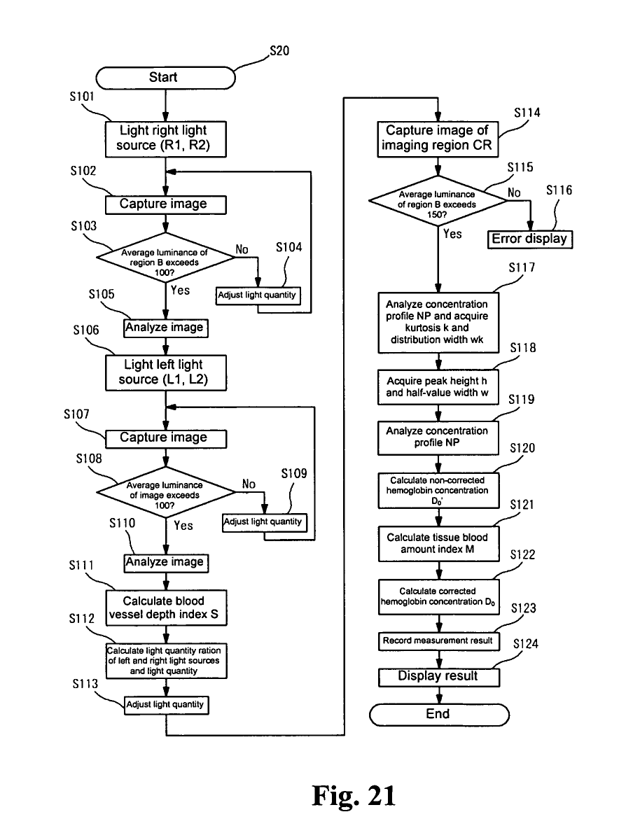

FIG. 21 is a flowchart showing the details of a measuring process of the hemoglobin concentration by a non-invasive blood component measuring device according to another embodiment. The processes of steps S101 to S118 in the flowchart are the same as the processes of steps S101 to S118 in the flowchart of FIG. 13, and thus the description on the portion redundant with the description in the flowchart of FIG. 13 will be omitted. The process after step S119 will be described below.

In the process of step S119, the CPU 53a analyzes the concentration profile NP created in step S117, and calculates the kurtosis (k) and the distribution width (dw) of the concentration profile NP.

The process then proceeds to step S120, and the CPU 53a calculates the non-corrected hemoglobin concentration D₀′ by the following formula (12), and stores the result in the frame memory 53a.

D₀′=h/[2{(k+α)/dw^{β}}/(π·w/2)]ⁿ (12)

The formula (12) is a formula for calculating the hemoglobin concentration non-dependent on the change in the blood vessel cross sectional shape, wherein an accurate hemoglobin concentration, taking the change in the blood vessel cross sectional shape into consideration, can be calculated without carrying out the step of obtaining the blood vessel cross sectional index N by using the formula (12). The formula (12) will be hereinafter described.

The CPU 53a calculates the tissue blood amount index M based on the blood vessel peripheral tissue in the image of the living body (step S121), calculates the corrected hemoglobin concentration D₀ based on the blood vessel depth index S and the tissue blood amount index M (step S122), records the measurement result (step S123), displays the result (step S124), and returns the process to the main routine.

The formula (12) will be described below.

In the first embodiment, the hemoglobin concentration is calculated with the half-value width w reflecting the blood vessel diameter in the direction orthogonal to the imaging direction replaced with the blood vessel diameter in the imaging direction under the assumption that the blood vessel cross section is a perfect circuit. If the hemoglobin concentration is calculated in this manner, the blood vessel diameter in the imaging direction and the blood vessel diameter in the direction orthogonal to the imaging direction do not match due to change in the blood vessel cross sectional shape, and the calculated hemoglobin concentration and the actual hemoglobin concentration sometimes deviate. In order to solve such problem, the first embodiment proposes a configuration of calculating the blood vessel cross sectional shape index N and correcting the hemoglobin concentration.

Therefore, if the hemoglobin concentration is calculated using the blood vessel diameter in the imaging direction in place of the half-value width w, the problem will not arise, and thus an accurate hemoglobin concentration non-dependent on the change in the blood vessel cross sectional shape can be calculated without carrying out the correction process. Assuming that the blood vessel diameter in the imaging direction is 2a, the hemoglobin concentration D₀′ based on the blood vessel diameter in the imaging direction is given by the following formula (13).

D₀′=h/(2a)ⁿ (13)

Solving formula (10), following formula (14) is obtained.

a={(k+α)/dw^{β}}/(π·w/2) (14)

Thus, according to formula (13) and formula (14), following formula (14) is obtained.

D₀′=h/[2{(k+α)/dw^{β}}/(π·w/2)]ⁿ (15)

Then, that is proved that formula (12) is logical.

From a different viewpoint, a different formula may be used as an formula for calculating the hemoglobin concentration.

The height h^{x} of the concentration profile NP at position X reflects the distance the light reaching position X has moved in the blood vessel, such that the peak height h of the concentration profile NP reflects the portion where the light entering the target blood vessel moves the longest distance, that is, the blood vessel diameter in the imaging direction. Similarly in the entire region of the distribution width of the concentration profile NP, the sum of the height hₓof the concentration profile NP corresponds to the sum of the distance the light moved in the blood vessel. The sum of the height hₓ is equal to the area of the concentration profile NP, and the sum of the distance the light moved in the blood vessel is equal to the cross sectional area of the blood vessel. Therefore, the formula (13) consisting of the ratio between the peak height of the concentration profile NP and the blood vessel diameter in the imaging direction can be replaced with the following formula.

D₀′=A/(Sa)ⁿ (16)

(In the formula, A is the area of the concentration profile NP)

The cross sectional area Sa of the blood vessel is obtained by formula (6). Therefore, following formula (17) is obtained by formula (16) and formula (6).

D₀′=A/[(k+α)/dw^{β}]ⁿ (17)

If formula (17) is used, the hemoglobin concentration D₀′ is given based on the cross sectional area of the blood vessel. Since the cross sectional area of the blood vessel is always constant even if the shape of the blood vessel changes, an accurate hemoglobin concentration non-dependent on the change in the blood vessel cross sectional shape can be calculated. As a still variant of the second embodiment, the formula (17) may be used in step S118 of the flowchart shown in FIG. 21.

What is claimed is:

1. A non-invasive blood component measuring device comprising:
a light source section for irradiating a light to a blood vessel through a skin;
an imaging section for imaging the irradiated blood vessel through the skin; and
a controller, including a memory under control of a processor, the memory storing instructions enabling the processor to carry out operations, comprising:
creating a concentration profile based on an image obtained by imaging the blood vessel with the imaging section;
calculating a blood component concentration based on the concentration profile;
acquiring a shape feature of the concentration profile; and
correcting the blood component concentration based on the shape feature of the concentration profile.

2. The non-invasive blood component measuring device of claim 1 wherein, the shape feature of the concentration profile includes a kurtosis of the concentration profile, and a distribution width at a predetermined height of the concentration profile.

3. The non-invasive blood component measuring device of claim 1 wherein,
the operations further comprise acquiring a blood vessel peripheral tissue blood amount based on a blood vessel peripheral tissue in the image; wherein,
the correcting operation is performed based on the shape feature of the concentration profile, and the blood vessel peripheral tissue blood amount.

4. The non-invasive blood component measuring device of claim 3 wherein, the operations further comprise:
creating a blood vessel depth profile based on the image obtained by imaging with the imaging section; and
acquiring a blood vessel depth based on the blood vessel depth profile; wherein,
the correcting operation is performed based on the shape feature of the concentration profile, the blood vessel peripheral tissue blood amount, and the blood vessel depth.

5. The non-invasive blood component measuring device of claim 1, wherein the calculating operation is performed based on a peak height of the concentration profile, and a distribution width at a predetermined height of the concentration profile.

6. The non-invasive blood component measuring device of claim 1 wherein, the blood component concentration is hemoglobin concentration.

7. A non-invasive blood component measuring device comprising:
a light source section for irradiating a light to a blood vessel through a skin;
an imaging section for imaging the irradiated blood vessel through the skin; and
a controller, including a memory under control of a processor, the memory storing instructions enabling the processor to carry out operations, comprising:
creating a concentration profile based on an image obtained by imaging the blood vessel with the imaging section; and
calculating a blood component concentration based on a peak height of the concentration profile, and a shape feature of the concentration profile.

8. The non-invasive blood component measuring device of claim 7 wherein, the shape feature of the concentration profile includes a kurtosis of the concentration profile, and a distribution width at a predetermined height of the concentration profile.

9. The non-invasive blood component measuring device of claim 7 wherein, the operations further comprise:
acquiring a blood vessel peripheral tissue blood amount based on a blood vessel peripheral tissue in the image; and
correcting the blood component concentration based on the blood vessel peripheral tissue blood amount.

10. The non-invasive blood component measuring device of claim 9 wherein, the operations further comprise:
creating a blood vessel depth profile based on the image obtained by imaging with the imaging section; and
acquiring a blood vessel depth based on the blood vessel depth profile; wherein,
the correcting operation is performed based on the blood vessel peripheral tissue blood amount and the blood vessel depth.

11. The non-invasive blood component measuring device of claim 7 wherein the blood component concentration is hemoglobin concentration.

12. A non-invasive blood component measuring method comprising the steps of:
irradiating a light to a blood vessel through a skin and imaging the irradiated blood vessel through the skin;
creating a concentration profile distributed across the blood vessel based on an image obtained by imaging the blood vessel;
calculating a blood component concentration based on the concentration profile;
acquiring a shape feature of the concentration profile; and
correcting the blood component concentration based on the shape feature of the concentration profile.

13. The non-invasive blood component measuring method of claim 12, wherein the shape feature of the concentration profile includes a kurtosis of the concentration profile, and a distribution width at a predetermined height of the concentration profile.

14. The non-invasive blood component measuring method of claim 12, further comprising a step of acquiring a blood vessel peripheral tissue blood amount based on a blood vessel peripheral tissue in the image, and
the correcting step is performed based on the shape feature of the concentration profile and the blood vessel peripheral tissue blood amount.

15. The non-invasive blood component measuring method of claim 14, further comprising the steps of:
creating a blood vessel depth profile based on the image obtained by imaging the blood vessel; and
acquiring a blood vessel depth based on the blood vessel depth profile; and
the correcting step is performed based on the shape feature of the concentration profile, the blood vessel peripheral tissue blood amount, and the blood vessel depth.

16. The non-invasive blood component measuring method of claim 12, wherein the calculating step is performed based on a peak height of the concentration profile, and a distribution width at a predetermined height of the concentration profile.

17. A non-invasive blood component measuring method comprising the steps of:
irradiating a light to a blood vessel through a skin and imaging the irradiated blood vessel through the skin;
creating a concentration profile distributed across the blood vessel based on an image obtained by imaging the blood vessel; and
calculating a blood component concentration based on a peak height of the concentration profile and a shape feature of the concentration profile.

18. The non-invasive blood component measuring method of claim 17, wherein the shape feature of the concentration profile includes a kurtosis of the concentration profile, and a distribution width at a predetermined height of the concentration profile.

19. The non-invasive blood component measuring method of claim 17, further comprising the steps of:
acquiring a blood vessel peripheral tissue blood amount based on a blood vessel peripheral tissue in the image; and
correcting the blood component concentration based on the blood vessel peripheral tissue blood amount.

20. The non-invasive blood component measuring method of claim 19, further comprising the steps of:
creating a blood vessel depth profile based on the image obtained by imaging the blood vessel; and
acquiring a blood vessel depth based on the blood vessel depth profile; and
the correcting step is performed based on the blood vessel peripheral tissue blood amount and the blood vessel depth.
